(19) United States
(12) Patent Application Publication
(10) Pub. No.: US 2006/0247789 A1
(43) Pub. Date: Nov. 2, 2006

(54) Method and device for stabilization of prosthetic devices

(75) Inventors: Carlos Gil, Collierville, TN (US); Randall Allard, Germantown, TN (US); Greg Marik, Germantown, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/118,731

(22) Filed: Apr. 29, 2005

(51) Int. Cl. A61F 2/28; A61F 2/44

(52) U.S. Cl. 623/23.5; 623/17.11

(57) ABSTRACT

Methods and devices for providing a stabilized implant at a site of implantation include providing locking members on faces of the implant and applying bone cement to the implant faces. The locking members secure the bone cement so that the bone cement will not be substantially displaced when the implant is inserted at an implantation site.

### BACKGROUND

The present disclosure relates generally to methods and devices for stabilizing prosthetic devices at a site of implantation. According to one example, prosthetic devices implanted between vertebrae are stabilized therein with bone cement.

Spinal discs, which extend between the endplates of adjacent vertebrae in a spinal column of the human body, can rupture, degenerate and/or protrude by injury, degradation, disease or the like. In some cases, surgical correction may be required.

Surgical correction can include removal of the spinal disc from between the adjacent vertebrae, and insertion of a prosthetic device between the adjacent vertebrae. In this context, prosthetic devices may be referred to as intervertebral prosthetic joints, prosthetic implants, disc prostheses or artificial discs, among other labels.

For stabilization between the vertebrae, such prosthetic devices currently use surface modification, for example, glass beads or plasma spray, to promote ingrowth of bone or fibrous tissue. The bony endplates of the adjacent vertebrae can also be prepared in such a manner that promotes stability, for example, the endplates can be milled to have a shape corresponding to that of the implant.

### SUMMARY OF THE INVENTION

In an exemplary embodiment, an implant that can be stabilized at a site of implantation is provided. An exemplary implant includes a top portion having a top implant face, a bottom portion having a bottom implant face, and a plurality of locking members formed on at least one of the top implant face and the bottom implant face. The locking members provide a matrix for substantially securing bone cement applied thereto. The bone cement will be adjacent to an implantation surface upon insertion at an implantation site, and will thus provide for stabilization and fixation of the implant at the implantation site.

In another exemplary embodiment, a method of stabilizing an implant at an implantation site is provided. An exemplary method includes providing an implant having at least one face comprising a plurality of locking members. The method further includes applying bone cement to the face of the implant having the locking members, and inserting the implant at the implantation site such that the face having the bone cement applied thereto is adjacent to an implantation surface that defines the implantation site at least in part. Thus, the bone cement provides for stabilization and fixation of the implant at the implantation site.

In yet another exemplary embodiment, a system for fixing an implant at an implantation site is provided. The system includes an implant having means for securing bone cement to at least one face of the implant, a source of bone cement, and means for inserting the implant at the implantation site such that the face having the bone cement applied thereto is adjacent to an implantation surface that defines the implantation site at least in part. Thus, the bone cement provides for stabilization and fixation of the implant at the implantation site.

### BRIEF DESCRIPTION OF DRAWINGS

The disclosure can be more clearly understood by reference to the following drawings, which illustrate exemplary embodiments thereof, and which are not intended to limit the scope of the appended claims.

FIG. 1 is a perspective view of an exemplary embodiment of an intervertebral prosthetic device having a plurality of cement-locking members formed on top and bottom faces.

FIG. 2 is an end view of the intervertebral prosthetic device shown in FIG. 1.

FIG. 3 is an isolated view of a cement-locking member of the type illustrated in FIG. 1.

FIGS. 4A-4F are isolated views of alternative embodiments of cement-locking members.

FIG. 5 is an end view of the intervertebral prosthetic device shown in FIG. 1, with bone cement applied to respective faces.

FIG. 6 is a sectional view of an intervertebral prosthetic device implanted between two vertebrae.

FIG. 7 is a sectional view of another intervertebral prosthetic device implanted between two vertebrae.

The disclosure can be more clearly understood by reference to some of its specific embodiments, described in detail below, which description is not intended to limit the scope of the claims in any way.

### DETAILED DESCRIPTION

Methods and devices as described herein can provide for stabilization of an implant at an implantation site, for example, between adjacent vertebrae. Methods and devices as described herein can provide for fixation of an implant to an implantation surface that defines an implantation site at least in part, for example, an implantation site between adjacent vertebrae defined in part by an implant surface comprising a vertebral endplate of one of the vertebrae.

Methods and devices as described herein provide a cement mantle on an implant face adjacent to an implantation surface that defines an implantation site at least in part, for example, an implantation site between adjacent vertebrae defined in part by an implantation surface comprising a vertebral endplate of one of the vertebrae. In certain examples, methods and devices as described herein provide a substantially uniform cement mantle on an implant face, promote stabilization of the implant and promote fixation of the implant at an implantation site. Certain examples also can provide for a substantially uniform cement mantle having a minimized thickness, for example, less than 4 millimeters.

Implants as described herein can be used as implants in the spine. The description below focuses on an exemplary embodiment wherein the implant is a spinal disc endoprosthesis. The methods and features described herein could be used with implants for use in other spinal joints. In the description which follows, like parts are marked throughout the specification and drawings with the same reference numerals, respectively. The drawing figures are not necessarily to scale in the interest of clarity and conciseness.

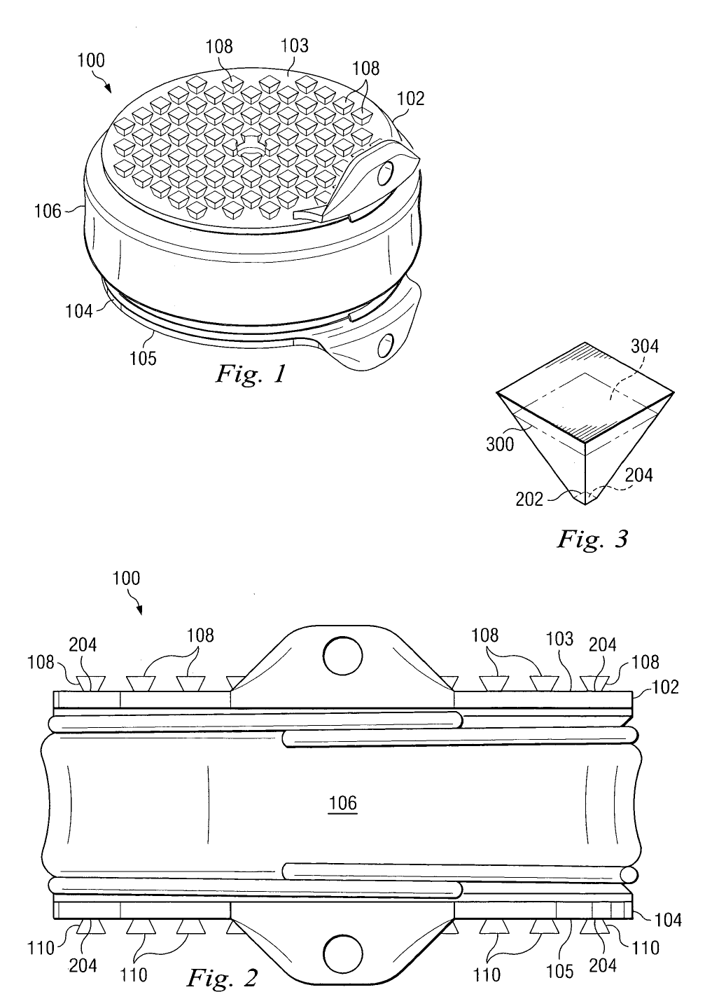

According to the exemplary embodiment illustrated in FIGS. 1 and 2, an implant 100 comprises a top portion 102, a bottom portion 104 and a middle portion 106. The top portion 102 and bottom portion 104 each comprises an implant face 103 and 105, respectively, upon which a plurality of locking members can be formed. Each implant face 103 and 105 will be adjacent to an implantation surface that defines an implantation site at least in part, for example, a vertebral endplate between adjacent vertebrae. In the exemplary embodiment illustrated in FIGS. 1 and 2, a plurality of locking members 108 and 110 are illustrated on the implant face of each of top portion 102 and bottom portion 104, respectively (110 being visible in FIG. 2). In other embodiments, however, locking members can be on the face of either the top portion or the bottom portion.

Locking members 108 and 110 provide a matrix for securing a cement mantle to the top and bottom portions 102, 104 of the implant 100. In the exemplary embodiment illustrated in FIG. 1, locking members 108 and 110 comprise pyramid structures secured to the implant faces 103, 105, at a contact area 204, which is best viewed in FIG. 2. Contact area 204 lies in the plane defined by contact of the locking member 108, 110 with the face 103, 105 of the implant.

FIG. 3 is an isolated view of any one of the locking members 108, 110 illustrated in FIGS. 1 and 2. Contact area 204 is the area through the locking member lying in the contact plane 202, in which the locking member will contact the implant face. A distal area 304 is defined by a plane 300 through the locking member, which plane 300 is substantially parallel to and distal from the contact plane 202, such that the distal area 304 will be larger than the contact area 204.

In FIG. 3, a distal plane 300 substantially parallel to the contact plane 202 is illustrated as located distal from the contact plane 202 around the base of the pyramid. Distal plane 300 through the locking member defines a distal area 304 that is larger than the contact area 204. Plane 300 need not be located around the base of the pyramid, rather plane 300 can be located at any point distal from the contact plane 202 that would result in an area defined by the plane 300 through the locking member that is larger than the contact area 204.

Referring again to the exemplary embodiment illustrated in FIGS. 1 and 2, the middle portion 106 can be one or more components, can be a component separate from the top and bottom portions, or can be a component integral with the top portion, or the bottom portion, or both. In those examples where the middle portion is integral with the top portion, the bottom portion, or both, then it may not be referred to as a middle portion, but rather, would be included with any reference to that portion with which it is integral. In those examples where the middle portion is a separate component from the top or the bottom portions, the middle portion may articulate with one or both of the top and bottom portions. Articulating spinal implants having faces that will be adjacent to an implantation surface upon insertion at an implantation site, and upon which locking members as described herein can be formed, are known to those of ordinary skill in the art, and include but are not limited to those illustrated in U.S. Patent Publication Nos. 2002/0128715, 2003/0135277, 2004/0225366 and 2004/0158328.

Other spinal implants having faces that will be adjacent to an implantation surface upon insertion at an implantation site, and upon which locking members as described herein can be formed, include those described in U.S. Pat. Nos. 5,674,296, 5,865,846, 6,156,067 and 6,001,130. A variety of other spinal implants can also be used, so long as the implant has at least one face that will be adjacent to an implantation surface upon insertion at an implantation site, and upon which a plurality of locking members as described herein can be formed.

The components of the implant, in particular, the top and bottom portions 102, 104 may be constructed of polyethylene or other suitable polymeric material. The locking members may be made of the same material used to make the top or bottom portion of the implant, or may be of a different material. According to one example, the locking members are made of bone cement, which may be the same cement that will be applied to the implant face prior to insertion. Providing locking members made of bone cement allows the implant face to provide a uniform cement mantle for stabilizing the implant and securing the implant to the implantation surface.

The locking members can be machined, molded, or added onto one or more faces of the top and bottom portions during or after the manufacture of the top or bottom portions. In certain examples, the locking members can be formed by molding bone cement on the faces of the top and bottom portions of the implant, and allowing the cement to cure. In other examples, the locking members can be molded from bone cement, and then inserted into mated receptacles formed on the implant face. Such receptacles can be machined onto the implant face in a shape complementary to the locking member so as to secure the locking member therein. Locking members 108, 110 can also be formed on the implant face such that a portion of the locking member extends below the plane in which the locking member contacts the implant face. In such embodiments, therefore, a portion of the locking member extends into the body of the implant such that a portion of the locking member will be below the contact area.

In certain examples, the locking members are formed to have a height that provides a cement mantle of a minimal thickness on the implant face. For example, the locking members can have a height of less than about 4 millimeters, as measured on a perpendicular to the implant face. In other examples, the locking members can have a height of about 1, about 2, or about 3 millimeters. The height of the locking members helps to control the height of the cement mantle formed on the implant face. The height at which to form the locking member can be determined by the type of implant the locking member will be used with, the location and parameters of the implant site, or a variety of other factors. Selection of a desired height can thus be determined by one of ordinary skill in the art through routine experimentation.

Locking members 108, 110 can be machined, molded, or otherwise formed in any shape and size that can be positioned on the faces of top and bottom portions 102, 104 such that an area defined by a plane through the locking member, substantially parallel to and distal from the implant face, is greater than the area defined by the plane in which the locking member contacts the implant face.

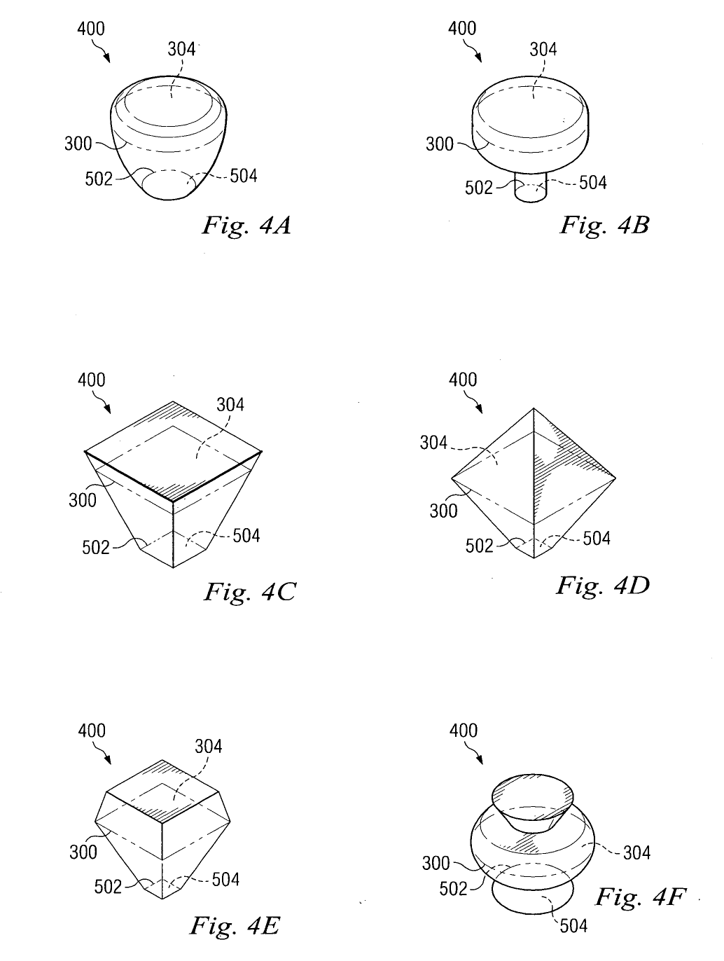

If a pyramid shape is used for locking members, such as the shape illustrated in FIGS. 1-3, the sides of the pyramid can be in the shape of isosceles, scalene or equilateral triangles. Several other exemplary shapes for locking members are illustrated in FIGS. 4A-4F.

The parachute-shaped locking member 400 illustrated in FIG. 4A has a contact area 504 lying in the contact plane 502 in which the locking member will contact an implant face. A distal plane 300 through the locking member, lying substantially parallel to and distal from the contact plane 502, defines a distal area 304. The plane 300 is located at a point distal from the contact area 504 such that the distal area 304 is greater than the contact area.

The thumb tack-shaped locking member 400 illustrated in FIG. 4B has a contact area 504 lying in the contact plane 502 in which the locking member will contact an implant face. A distal plane 300 through the locking member, lying substantially parallel to and distal from the contact plane 502, defines a distal area 304. The plane 300 is located at a point distal from the contact area 504 such that the distal area 304 is greater than the contact area.

The frusto-conical shaped locking member 400 illustrated in FIG. 4C has a contact area 504 lying in the contact plane 502 in which the locking member will contact an implant face. A distal plane 300 through the locking member, lying substantially parallel to and distal from the contact plane 502, defines a distal area 304. The plane 300 is located at a point distal from the contact area 504 such that the distal area 304 is greater than the contact area.

The rhombus-shaped locking member 400 illustrated in FIG. 4D has a contact area 504 lying in the contact plane 502 in which the locking member will contact an implant face. A distal plane 300 through the locking member, lying substantially parallel to and distal from the contact plane 502, defines a distal area 304. The plane 300 is located at a point distal from the contact area 504 such that the distal area 304 is greater than the contact area.

The diamond-shaped locking member 400 illustrated in FIG. 4E has a contact area 504 lying in the contact plane 502 in which the locking member will contact an implant face. A distal plane 300 through the locking member, lying substantially parallel to and distal from the contact plane 502, defines a distal area 304. The plane 300 is located at a point distal from the contact area 504 such that the distal area 304 is greater than the contact area.

The clover-shaped locking member 400 illustrated in FIG. 4F has a contact area 504 lying in the contact plane 502 in which the locking member will contact an implant face. A distal plane 300 through the locking member, lying substantially parallel to and distal from the contact plane 502, defines a distal area 304. The plane 300 is located at a point distal from the contact area 504 such that the distal area 304 is greater than the contact area.

In each of FIGS. 4A-4F, the plane 300 (and therefore the distal area 304) is illustrated as located around a point distal from-the contact area 504 that causes the distal area 304 to have approximately the maximum area dictated by the shape of the particular locking member. However, the plane 500 could be located at any point distal from the contact area 504 that would result in the distal area 304 being larger than the contact area.

The locking members can have other shapes not illustrated herein, so long as the shape provides a plane through the locking member, located substantially parallel to a contact plane, which at some point distal from the contact plane will define an area that is greater than the contact area. In addition, the individual members comprising the plurality of locking members can have the same or different shapes.

As described above, each locking member has a shape such that a contact area is defined in a plane in which the locking member contacts the face of the implant, and a distal area is defined in a plane substantially parallel to the contact plane and located at some point distal therefrom, such that the distal area is greater than the contact area. This design provides a matrix that “locks” bone cement applied to the face of the implant so that the cement will not be substantially displaced when the implant is inserted at an implantation site, for example, when the implant is inserted between adjacent vertebrae. The “locked” bone cement provides a substantially stable cement mantle, so that there is better opportunity for adhesion to an implantation surface, for example, a vertebral endplate, and stabilization of the implant at the implantation site, for example, between adjacent vertebrae.

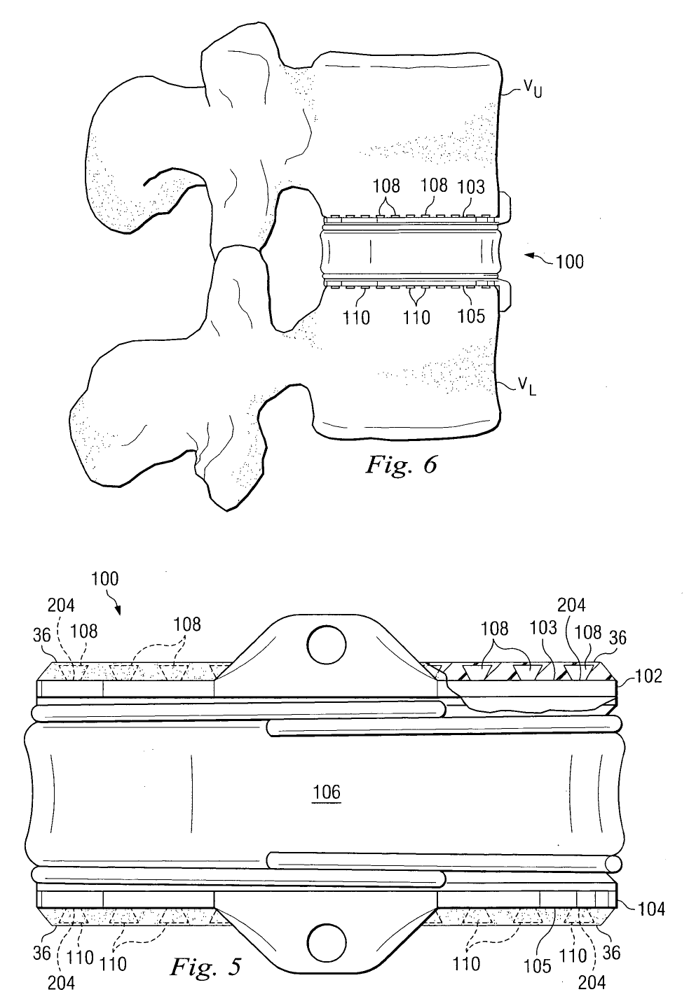

According to an exemplary method for inserting an implant as described herein at an implantation site, bone cement is applied to the face of the implant that has locking members prior to insertion of the implant at the implantation site. In the exemplary embodiment illustrated in FIG. 5, the faces 103, 105 of each of the top and bottom portions 102, 104, of the implant have locking members 108, 110 formed thereon. Bone cement 36 can be applied to the implant in a dough-like state, which is achieved by allowing a viscous bone cement to cure, for example for 1 minute, 2 minutes, 4 minutes, 6 minutes, or more, before application to the implant. Alternatively, bone cement 36 can be applied to the implant in a viscous state, and allowed to cure slightly on the implant, prior to insertion between adjacent vertebrae.

Bone cement 36 is applied to the implant in an amount such that at least a portion of the cement will enter the matrix formed by the locking members, and in particular, will enter that area of the matrix around the contact areas. The amount of bone cement will also be such that a cement mantle having a height substantially the same as the height of the locking members, as measured perpendicular to the implant face, is formed. A portion of the cement may also reside on surfaces of the locking members that will be adjacent to an implantation surface that defines the implantation site at least in part. For example, vertebral endplates can provide an implantation surface that defines an implantation site at least in part.

Upon insertion between adjacent vertebrae, some amount of the bone cement residing on the surfaces of the locking members may be displaced. However, the matrix provided by the locking members will hold an amount of the cement in place, thus providing a cement mantle of approximately the same height, measured on a perpendicular to the implant face, as the locking members. Thus, the height of the locking members can be used to control the thickness of the cement mantle, and provide a cement mantle of a minimal thickness, for example, less than 4 millimeters, as measured perpendicular to the implant face.

The cement mantle allows for at least a portion of the cement to come into contact with the endplates of the adjacent vertebrae upon insertion. Pressure from the endplates of the respective vertebrae upon the respective top and bottom portions of the implant may also press the cement further into the matrix comprised by the locking members. In addition, the implant may be urged against the vertebral endplates until the cement cures. Any of a variety of instruments known to those of ordinary skill in the art are suitable for urging the implant against the vertebral endplates.

The bone cement can be applied with devices and methods known to those of ordinary skill in the art. No particular method or device is required, thus any source of bone cement is suitable for use with the methods and implants described herein.

Polymethylmethcrylate (PMMA) or other bone cements known to those of ordinary skill in the art are suitable for use with the methods and devices described herein. Further, antibiotics could be added to the cement to reduce the risk of infection.

Referring now to FIG. 6, an implant 100 is illustrated at an implantation site between adjacent vertebrae V_{U} and V_{L}. The implant 1 can be of the type illustrated in FIG. 1, or can be any other type of implant that can provide at least one surface 103 or 105 that will be adjacent to a vertebral endplate upon insertion, and upon which locking members can be formed. In the embodiment illustrated in FIG. 6, locking members 108, 110 are formed on both surfaces 103, 105 of the implant. Bone cement can be applied to one or both of the surfaces 103, 105, and will be secured there by the locking members 108, 110.

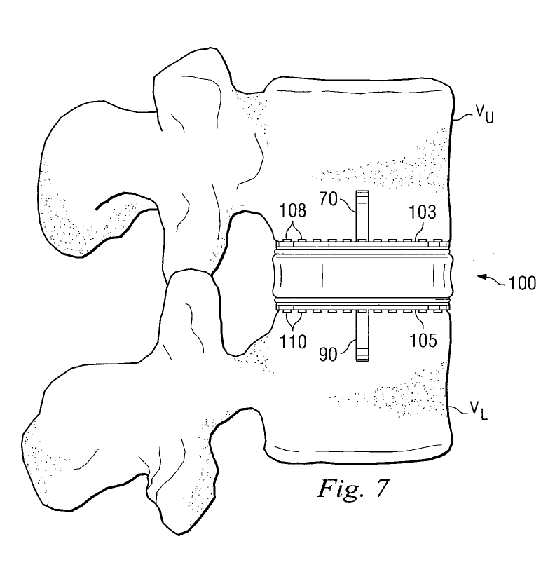

FIG. 7 illustrates another example of implant 100 having locking members formed on at least one surface 103 or 105. The locking members will provide a matrix for securing a cement mantle to the implant surface. In the embodiment illustrated in FIG. 7, the implant has tabs 70, 90, which extend into bores prepared in the vertebral endplates. The tabs and the cement mantle provided by the locking members on the implant surface provide a system for stabilizing the implant between the vertebrae. Preparation of the vertebral endplates, for example forming bores as illustrated in FIG. 7, is optional, and may be used depending on the type of implant to be inserted.

In certain examples, the endplates of the vertebrae can be prepared by milling or cutting the surfaces. The endplates can also be shaped to correspond at least in part to the shape of the implant face. Holes could also be drilled through the endplates of the vertebrae. Hypotensive anesthesia and hemostatic agents could also be used to prepare the vertebral endplates. Hypotensive anesthesia and hemostatic agents may help reduce bleeding from the vertebral endplates. In certain examples, the endplate surface will be as dry as possible when the implant is inserted.

Where the implant is used as a spinal disc endoprosthesis inserted between two adjacent vertebral bodies, the implant may be introduced using an anterior approach. The implanting procedure is carried out after discectomy, according to methods known to those of ordinary skill in the art. The appropriate size of the implant for a particular patient, determination of the appropriate location of the implant in the intervertebral space, and implantation in the intervertebral space are all desirably accomplished using precision stereotactic techniques, apparatus, and procedures, such as the techniques and procedures known to those of ordinary skill in the art. Non-stereotactic techniques can also be used. Further, implants can be produced in a variety and range of sizes.

The invention has been described above with respect to certain specific embodiments thereof. Those of skill in the art will understand that variations from these specific embodiments that are within the spirit of the invention will fall within the scope of the appended claims and equivalents thereto.

What is claimed is:

1. An implant comprising:
a top portion having a top implant face;
a bottom portion having a bottom implant face;
a plurality of locking members formed on at least one the top implant face and the bottom implant face, which locking members provide a matrix for substantially securing bone cement applied thereto.

2. The implant of claim 1 wherein the plurality of locking members have a shape such that:
a contact plane through the locking members where the locking members contact the face of the implant defines a contact area of the locking member; and
a distal plane through the locking member, located substantially parallel and distal from the contact plane, defines a distal area that is larger than the contact area.

3. The implant of claim 2 wherein the shape of the locking members is at least one shape selected from the group consisting of pyramid, parachute, thumb tack, frusto-conical, rhombus, diamond and clover.

4. The implant of claim 1 wherein the locking members have a height of less than about 4 millimeters, as measured perpendicular to the implant face.

5. The implant of claim 1 wherein:
the top implant face has a plurality of locking members formed thereon; and
the bottom implant face has a plurality of locking members formed thereon, and wherein:
the locking members on each of the top implant face and the bottom implant face have a shape such that a contact plane through the locking members where the locking members contact the face of the implant defines a contact area of the locking member; and a distal plane through the locking member, located at a point substantially parallel to and distal from the contact area, defines a distal area that is larger than the contact area.

6. The implant of claim 5 wherein the locking members on the top implant face have the same shape as the locking members on the bottom implant face.

7. The implant of claim 5 wherein the locking members on the top implant face have a different shape than the locking members on the bottom implant face.

8. The implant of claim 1 wherein the locking members are formed on the implant face by at least one of machining, molding and mating.

9. The implant of claim 1 wherein the locking members comprise bone cement.

10. The implant of claim 1 wherein the bone cement comprises polymethylmethacrylate.

11. The implant of claim 10 wherein the locking members comprise polymethylmethacrylate.

12. The implant of claim 1 further comprising:
a middle portion contacting the top portion and the bottom portion, which middle portion articulates with at least one of the top portion and the bottom portion.

13. The implant of claim 1 further comprising:
tabs that will extend into bores in the implantation surface.

14. A method of stabilizing an implant at an implantation site comprising:
providing an implant having at least one face comprising a plurality of locking members;
applying bone cement to the face of the implant having the locking members; and
inserting the implant at the implantation site such that the face having the bone cement applied thereto is adjacent to an implantation surface that defines the implantation site at least in part.

15. The method of claim 14 wherein the locking members have a shape such that:
a contact plane through the locking members where the locking members contact the face of the implant defines a contact area of the locking member; and
a distal plane through the locking members, located substantially parallel to and distal from the contact plane, defines a distal area that is larger than the contact area.

16. The method of claim 15 wherein the shape of the locking members is at least one shape selected from the group consisting of pyramid, parachute, thumb tack, frusto-conical, rhombus, diamond and clover.

17. The method of claim 14 further comprising:
providing an implant having a top implant face with a plurality of locking members formed thereon, and a bottom implant face having a plurality of locking members formed thereon, wherein the locking members on each of the top implant face and the bottom implant face have a shape such that:
a contact plane through the locking members where the locking members contact the face of the implant defines a contact area of the locking member; and
a distal plane through the locking members, located substantially parallel to and distal from the contact plane, defines a distal area that is larger than the contact area.

18. The method of claim 14 wherein the bone cement comprises polymethylmethacrylate.

19. The method of claim 18 wherein the bone cement further comprises an antibiotic.

20. The method of claim 14 further comprising:
preparing the implantation surface with at least one of hypotensive anesthesia and a hemostatic agent prior to inserting the implant.

21. The method of claim 14 further comprising at least one of:
milling the implantation surface, cutting the implantation surface, forming a bore in the implantation surface and shaping the implantation surface.

22. A method for inserting an implant at an implantation site comprising:
providing an implant having at least one face comprising a plurality of locking members, which locking members comprise bone cement;
forming a cement mantle on the face of the implant having the locking members; and
inserting the implant at the implantation site such that the face having the bone cement applied thereto is adjacent to an implantation surface that defines the implantation site at least in part.

23. The method of claim 22 wherein the locking members have a shape such that:
a contact plane through the locking members where the locking members contact the face of the implant defines a contact area of the locking member; and
a distal plane through the locking members, located substantially parallel to and distal from the contact plane defines a distal area that is larger than the contact area.

24. The method of claim 23 wherein the shape of the locking members is at least one shape selected from the group consisting of pyramid, parachute, thumb tack, frusto-conical, rhombus, diamond and clover.

25. The method of claim 22 further comprising:
providing an implant having a top implant face with a plurality of locking members formed thereon, and a bottom implant face having a plurality of locking members formed thereon, wherein the locking members on each of the top implant face and the bottom implant face have a shape such that:
a contact plane through the locking members where the locking members contact the face of the implant defines a contact area of the locking member; and
a distal plane through the locking members, located substantially parallel to and distal from the contact plane, defines a distal area that is larger than the contact area.

26. The method of claim 22 wherein the locking members control the thickness of the cement-mantle.

27. The method of claim 26 wherein the thickness of the cement mantle is minimized.

28. The method of claim 26 wherein the cement mantle is less than about 4 millimeters thick, as measured on a perpendicular to the implant face.

29. The method of claim 22 wherein the locking members have a height, as measured perpendicular to the implant face, selected from the group consisting of: about 1 millimeter, about 2 millimeters, about 3 millimeters and about 4 millimeters.

30. The method of claim 29 wherein the cement mantle formed on the implant face has approximately the same height as the locking members.

31. A system for fixing an implant at an implantation site comprising:
an implant having means for securing bone cement to at least one face of the implant;
a source of bone cement; and
means for inserting the implant at the implantation site such that the face having the bone cement applied thereto is adjacent to an implantation surface that defines the implantation site at least in part.

32. The system of claim 31 further comprising:
means for securing bone cement to a top implant face; and
means for securing bone cement to a bottom implant face.

33. The system of claim 31 wherein the bone cement comprises polymethylmethacrylate.
